(19) United States
(12) Patent Application Publication
(10) Pub. No.: US 2022/0175273 A1
(43) Pub. Date: Jun. 9, 2022

(54) INFORMATION PROCESSING DEVICE, LOG ACQUISITION SYSTEM, ENERGY CALCULATION SYSTEM, INFORMATION PROCESSING METHOD, AND STORAGE MEDIUM

(71) Applicant: NEC Corporation, Minato-ku, Tokyo (JP)

(72) Inventors: Chenhui HUANG, Tokyo (JP); Kazuki IHARA, Tokyo (JP); Noriyuki TONOUCHI, Tokyo (JP); Kenichiro FUKUSHI, Tokyo (JP)

(73) Assignee: NEC Corporation, Minato-ku, Tokyo (JP)

(21) Appl. No.: 17/617,396

(22) PCT Filed: Jun. 12, 2019
(86) PCT No.: PCT/JP2019/023359

§ 371 Date: Dec. 8, 2021

(51) Int. Cl. A61B 5/103; A61B 5/11; A61B 5/00

(52) U.S. Cl. CPC A61B 5/1036; A61B 5/1123; A61B 5/4866; A61B 5/6807; A61B 5/7257; A61B 5/1118

(57) ABSTRACT

Provided is an information processing device including an action information acquisition unit configured to acquire action information of a user, a pedaling period extracting unit configured to extract a plurality of pedaling periods during which the user pedals a bicycle from the action information, and a calculation unit configured to calculate a non-pedaling time during which the user is on the bicycle and does not pedal based on a start time of each of the plurality of pedaling periods and an end time of each of the plurality of pedaling periods.

### TECHNICAL FIELD

The present invention relates to an information processing device, a log acquisition system, an energy calculation system, an information processing method, and a storage medium.

### BACKGROUND ART

Patent Literature 1 discloses a device for determining a pose using an acceleration sensor mounted on a human body. The device of the Patent Literature 1 determines whether the person is walking, running, lying, sitting, or standing based on the three axial acceleration acquired by the acceleration sensor.

### CITATION LIST

### Patent Literature

PTL 1: Japanese Patent Application Laid-open No. 2010-125239

### Non Patent Literature

NPL 1: Sebastian O. H. Madgwick, Andrew J. L. Harrison, and Ravi Vaidyanathan, “Estimation of IMU and MARG orientation using a gradient descent algorithm,” 2011 IEEE International Conference on Rehabilitation Robotics, pp. 179-185, 2011.

### SUMMARY OF INVENTION

### Technical Problem

In a case where the pose determination method disclosed in Patent Literature 1 is applied to determination of a state of a user riding a bicycle, there is a possibility that various states during riding cannot be considered.

The present invention intends to provide an information processing device, a log acquisition system, an energy calculation system, an information processing method, and a storage medium which can appropriately determine a state of a user riding a bicycle.

### Solution to Problem

According to one example aspect of the invention, provided is an information processing device including an action information acquisition unit configured to acquire action information of a user, a pedaling period extracting unit configured to extract a plurality of pedaling periods during which the user pedals a bicycle from the action information, and a calculation unit configured to calculate a non-pedaling time during which the user is on the bicycle and does not pedal based on a start time of each of the plurality of pedaling periods and an end time of each of the plurality of pedaling periods.

According to another example aspect of the invention, provided is an information processing method including acquiring action information of a user, extracting a plurality of pedaling periods during which the user pedals a bicycle from the action information, and calculating a non-pedaling time during which the user is on the bicycle and does not pedal based on a start time of each of the plurality of pedaling periods and an end time of each of the plurality of pedaling periods.

According to another example aspect of the invention, provided is a storage medium storing a program that causes a computer to perform acquiring action information of a user, extracting a plurality of pedaling periods during which the user pedals a bicycle from the action information, and calculating a non-pedaling time during which the user is on the bicycle and does not pedal based on a start time of each of the plurality of pedaling periods and an end time of each of the plurality of pedaling periods.

### Advantageous Effects of Invention

According to the present invention, an information processing device, a log acquisition system, an energy calculation system, an information processing method, and a storage medium which can appropriately determine a state of a user riding a bicycle can be provided.

### BRIEF DESCRIPTION OF DRAWINGS

FIG. 1 is a schematic diagram illustrating a general configuration of a log acquisition system according to a first example embodiment.

FIG. 2 is a block diagram illustrating a hardware configuration of a log acquisition device according to the first example embodiment.

FIG. 3 is a block diagram illustrating a hardware configuration of an information communication terminal according to the first example embodiment.

FIG. 4 is a functional block diagram of an information processing device according to the first example embodiment.

FIG. 5 is a flowchart illustrating an example of a log acquisition process performed by the log acquisition device according to the first example embodiment.

FIG. 6 is a flowchart illustrating an example of an extraction process of a pedaling period.

FIG. 7 is a flowchart illustrating an example of a pedaling state determination.

FIG. 8 is a graph illustrating an example of time series data of acceleration in the pedaling state.

FIG. 9 is a graph illustrating an example of time series data of the angle between a sole and ground in the pedaling state.

FIG. 10 is a graph illustrating an example of time series data of acceleration and time series data of angle when the user is walking.

FIG. 11 is a graph illustrating an example of frequency spectrum of the acceleration and frequency spectrum of the angle when the user is walking.

FIG. 12 is a graph illustrating an example of time series data of the acceleration and time series data of the angle in the pedaling state.

FIG. 13 is a graph illustrating an example of frequency spectrum of the acceleration and frequency spectrum of the angle in the pedaling state.

FIG. 14 is a diagram illustrating an example of a relation between a pedaling state and an identifier.

FIG. 15 is a graph illustrating an example of time series data of acceleration and time series data of angle in the vicinity of the getting-off time.

FIG. 16 is a functional block diagram of an information processing device according to a second example embodiment.

FIG. 17 is a flowchart illustrating an example of an energy calculation process performed by the energy calculation unit according to the second example embodiment.

FIG. 18 is a functional block diagram of an information processing device according to a third example embodiment.

FIG. 19 is a functional block diagram of an information processing device according to a fourth example embodiment.

Exemplary embodiments of the present invention are described below with reference to the drawings. Throughout the drawings, the same components or corresponding components are labeled with same references, and the description thereof may be omitted or simplified.

### FIRST EXAMPLE EMBODIMENT

A log acquisition system according to the present example embodiment is described. As a part of health management, there is a need to acquire a log of action including exercises such as daily walking time, bicycle riding time, and the like. The log acquisition system of the present example embodiment is a system for acquiring a log of action of a user including riding of a bicycle (action information).

The operation of the bicycle is broadly divided into two states, that is, a pedaling state in which the user is pedaling the bicycle and a non-pedaling state in which the user is not pedaling the bicycle. The exercise intensity is greatly different between the pedaling state and the non-pedaling state. Therefore, it is possible to acquire an effective log for managing exercise intensity by not only simply recording whether or not the user is riding a bicycle as a log, but also recording the time of each state by dividing the pedaling state and the non-pedaling state. In view of the above-described circumstances, the log acquisition system of the present example embodiment has a function of calculating a length of a period of a non-pedaling state (non-pedaling time).

The non-pedaling state is described more specifically. In recent years, a bicycle which is commercially available has been provided with a freewheel mechanism so that the bicycle can be traveled by inertia without turning a pedal. In such riding of the bicycle, a state in which the bicycle is traveling with inertia without pedaling by the user is included in the non-pedaling state. Further, the non-pedaling state includes a state in which the user is not pedaling in the operation of a bicycle equipped with a motor, which is provided with both a pedal and a motor such as a moped and is capable of traveling with human power.

In this specification, the number of wheels included in the bicycle is not particularly limited, and the “bicycle” may include not only a two-wheel bicycle but also a three-wheel bicycle, a bicycle with an auxiliary wheel, and the like. Further, even a vehicle equipped with a motor such as an electrically assisted bicycle or a bicycle with a motor is included in “bicycle” as long as it is provided with a mechanism capable of being driven by a pedal with human power. Further, the “bicycle” includes a stationary bicycle such as a bicycle for indoor training having a pedal like a two-wheel bicycle.

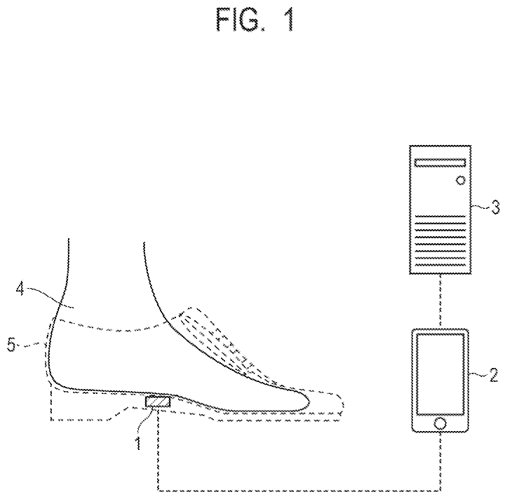

FIG. 1 is a schematic diagram illustrating a general configuration of a log acquisition system according to the present example embodiment. The log acquisition system includes a log acquisition device 1, an information communication terminal 2, and a server 3 which can be connected to each other by wireless communication.

The log acquisition device 1 is provided to be close to the sole of a shoe 5 worn by a user 4, for example. The log acquisition device 1 is an electronic apparatus having a sensing function for measuring a motion of the foot of the user 4, an information processing function for analyzing the measured motion information, a communication function with the information communication terminal 2, and the like. It is desirable that the log acquisition device 1 be provided at a position corresponding to the arch of the foot such as just below the arch of the foot. In this case, the log acquisition device 1 can measure acceleration and angular velocity of the center of the foot of the user 4. Since the center of the foot is a position showing the feature of the motion of the foot well, it is suitable for extracting features indicating the state of the user.

Note that, the log acquisition device 1 may be provided in the insole of the shoe 5, may be provided in the outsole of the shoe 5, or may be embedded in the shoe 5. The log acquisition device 1 may be detachably attached to the shoe 5 or may be non-detachably fixed to the shoe 5. The log acquisition device 1 may be provided at a portion other than the shoe 5 as long as the log acquisition device 1 can measure the motion of the foot. For example, the log acquisition device 1 may be provided in a sock which the user 4 is wearing, provided in a decoration, directly attached to the foot of the user 4, or embedded in the foot of the user 4. Although FIG. 1 illustrates an example in which one log acquisition device 1 is provided on one foot of the user 4, one log acquisition device 1 may be provided on each of both feet of the user 4. In this case, the motion information of both feet can be acquired in parallel, and more information can be acquired.

In this specification, the “foot” means a body part below an ankle of the user 4. In addition, in this specification, the “user” means a person who is an object of a determination of a process using the log acquisition device 1. Whether or not the user corresponds to the “user” is unrelated to whether or not the user is a user of a device other than the log acquisition device 1 constituting the log acquisition system, whether or not the user receives a service provided by the log acquisition system, or the like.

The information communication terminal 2 is a terminal device carried by the user 4, such as a cellular phone, a smartphone, or a smart watch. Application software for analyzing a state is installed in advance in the information communication terminal 2, and processing based on the application software is performed. The information communication terminal 2 acquires data acquired by the log acquisition device 1 from the log acquisition device 1 and performs information processing using the data. The result of the information processing may be notified to the user or may be transmitted to the server 3. The information communication terminal 2 may have a function of providing software such as a control program of the log acquisition device 1 or a data analysis program to the log acquisition device 1.

The server 3 provides application software for analyzing states to the information communication terminal 2 and updates the application software. The server 3 may store data acquired from the information communication terminal 2 and perform information processing using the data.

Note that, the general configuration is an example, and for example, the log acquisition device 1 may be directly connected to the server 3. Further, the log acquisition device 1 and the information communication terminal 2 may be configured as an integrated device, and another device such as an edge server or a relay device may be further included in the log acquisition system.

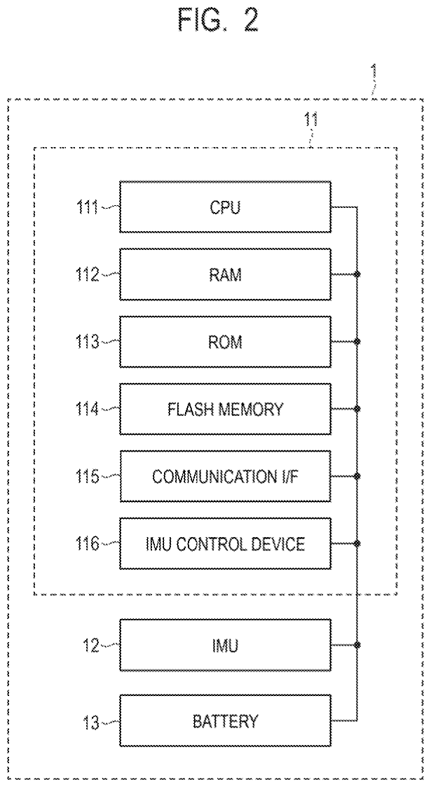

FIG. 2 is a block diagram illustrating a hardware configuration example of the log acquisition device 1. The log acquisition device 1 includes an information processing device 11, an inertial measurement unit (IMU) 12, and a battery 13.

The information processing device 11 is, for example, a microcomputer or a microcontroller that performs a control of the entire log acquisition device and data processing. The information processing device 11 includes a central processing unit (CPU) 111, a random access memory (RAM) 112, a read only memory (ROM) 113, a flash memory 114, a communication interface (I/F) 115, and an IMU control device 116. Each unit in the information processing device 11, the IMU 12, and the battery 13 is connected each other via a bus, wiring, a driving device, or the like.

The CPU 111 is a processor that performs predetermined calculation in accordance with a program stored in the ROM 113, the flash memory 114, or the like, and also has a function of controlling each unit of the information processing device 11. The RAM 112 is composed of a volatile storage medium and provides a temporary memory area required for the operation of the CPU 111. The ROM 113 is composed of a non-volatile storage medium and stores necessary information such as a program used for the operation of the information processing device 11. The flash memory 114 is a storage device composed of a non-volatile storage medium and temporarily storing data, storing an operation program of the information processing device 11, or the like.

The communication I/F 115 is a communication interface based on standards such as Bluetooth (registered trademark) or Wi-Fi (registered trademark), and is a module for performing communication with the information communication terminal 2.

The IMU 12 is a motion measurement device including an angular velocity sensor that measures angular velocity in three axial directions and an acceleration sensor that measures acceleration in three directions. The angular velocity sensor may be any sensor as long as it can acquire the angular velocity as time series data, and any type of sensor such as a vibration type sensor or a capacitance type sensor may be used. The acceleration sensor may be any type of sensor as long as it can acquire acceleration as time series data, and any type of sensor such as a piezoelectric type sensor, a piezoresistance type sensor, or a capacitance type sensor may be used. In the present example embodiment, the interval between the data points of the acquired time series data may be constant or may not be constant.

The IMU control device 116 is a control device that controls the IMU 12 to measure angular velocity and acceleration and acquires angular velocity and acceleration acquired by the IMU 12. The acquired angular velocity and acceleration are stored in the flash memory 114 as digital data. Note that analog-to-digital (AD) conversion for converting an analog signal measured by the IMU 12 into digital data may be performed in the IMU 12 or may be performed by the IMU control device 116.

The battery 13 is, for example, a secondary battery, and supplies power necessary for the operations of the information processing device 11 and the IMU 12. Since the battery 13 is built in the log acquisition device 1, the log acquisition device 1 can operate wirelessly without connecting to an external power source by wire.

Note that the hardware configuration illustrated in FIG. 2 is an example, and other devices may be added or some devices may not be provided. Further, some devices may be replaced by other devices having similar functions. For example, the information processing device 11 may further include an input device such as a button so that an operation by the user 4 can be accepted, and may further include an output device such as a display, a display lamp, and a speaker for providing information to the user 4. Thus, the hardware configuration illustrated in FIG. 2 can be changed appropriately.

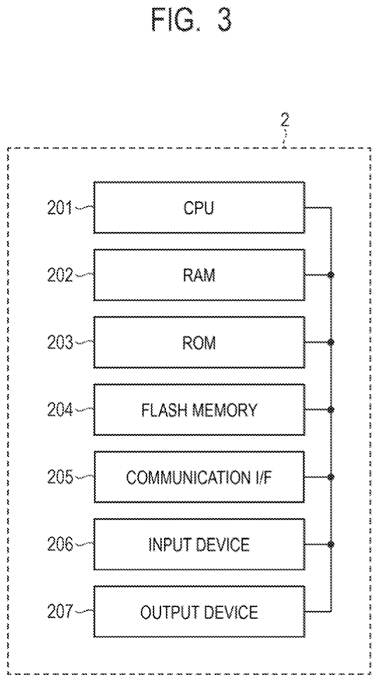

FIG. 3 is a block diagram illustrating a hardware configuration example of the information communication terminal 2. The information communication terminal 2 includes a CPU 201, a RAM 202, a ROM 203, and a flash memory 204. The information communication terminal 2 also includes a communication I/F 205, an input device 206, and an output device 207. Each unit of the information communication terminal 2 is connected to each other via a bus, wiring, a driving device, or the like.

In FIG. 3, each unit constituting the information communication terminal 2 is illustrated as an integrated device, but a part of these functions may be provided by an external device. For example, the input device 206 and the output device 207 may be external devices different from those constituting the functions of the computer including the CPU 201 or the like.

The CPU 201 is a processor that performs predetermined calculation in accordance with a program stored in the ROM 203, the flash memory 204, or the like, and also has a function of controlling each unit of the information communication terminal 2. The RAM 202 is composed of a volatile storage medium and provides a temporary memory area required for the operation of the CPU 201. The ROM 203 is composed of a non-volatile storage medium and stores necessary information such as a program used for the operation of the information communication terminal 2. The flash memory 204 is a storage device composed of a non-volatile storage medium for storing data transmitted and received to and from the log acquisition device 1 and for storing a program for operating the information communication terminal 2.

The communication I/F 205 is a communication interface based on standards such as Bluetooth (registered trademark), Wi-Fi (registered trademark), or 4G and is a module for performing communication with other devices.

The input device 206 is a user interface used by the user 4 to operate the information communication terminal 2. Examples of the input device 206 include a mouse, a trackball, a touch panel, a pen tablet, a button, or the like.

The output device 207 is, for example, a display device. The display device is a liquid crystal display, an organic light emitting diode (OLED) display, or the like, and is used for displaying information, displaying a graphical user interface (GUI) for operation input, or the like. The input device 206 and the output device 207 may be integrally formed as a touch panel.

Note that the hardware configuration illustrated in FIG. 3 is an example, and other devices may be added or some devices may not be provided. Further, some devices may be replaced by other devices having similar functions. Further, some functions of the present example embodiment may be provided by other devices via a network, or some functions of the present example embodiment may be realized by being distributed among a plurality of devices. For example, the flash memory 204 may be replaced by a hard disk drive (HDD) or a cloud storage. Thus, the hardware configuration illustrated in FIG. 3 can be changed appropriately.

The server 3 is a computer having substantially the same hardware configuration as that illustrated in FIG. 3. Since the hardware configuration of the server 3 is substantially the same as that of the information communication terminal 2 except that the server 3 may not be portable, a detailed description thereof is omitted.

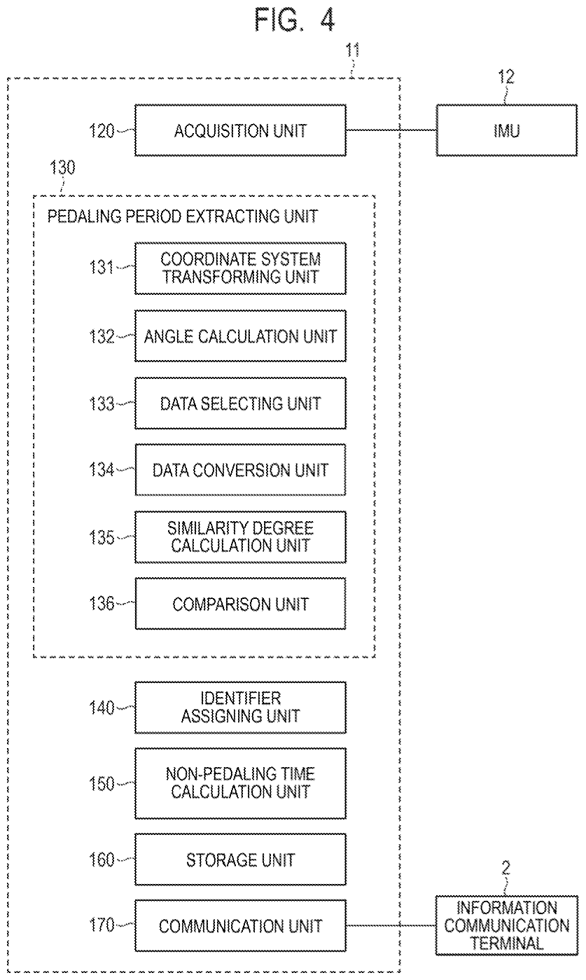

FIG. 4 is a functional block diagram of the information processing device 11 according to the present example embodiment. The information processing device 11 includes an acquisition unit 120, a pedaling period extracting unit 130, an identifier assigning unit 140, a non-pedaling time calculation unit 150, a storage unit 160, and a communication unit 170. The pedaling period extracting unit 130 includes a coordinate system transforming unit 131, an angle calculation unit 132, a data selecting unit 133, a data conversion unit 134, a similarity degree calculation unit 135, and a comparison unit 136.

The CPU 111 loads a program stored in the ROM 113, the flash memory 114, or the like into the RAM 112 and executes the program. Thus, the CPU 111 realizes the functions of the pedaling period extracting unit 130, the identifier assigning unit 140, and the non-pedaling time calculation unit 150. Further, the CPU 111 realizes the function of the acquisition unit 120 by controlling the IMU control device 116 based on the program. The CPU 111 realizes the function of the storage unit 160 by controlling the flash memory 114 based on the program. Further, the CPU 111 realizes the function of the communication unit 170 by controlling the communication I/F 115 based on the program. Specific processing performed by each of these units is described later.

In the present example embodiment, each function of the functional blocks illustrated in FIG. 4 is provided in the log acquisition device 1, but some functions of the functional blocks illustrated in FIG. may be provided in the information communication terminal 2 or the server 3. That is, the above-described functions may be realized by any of the log acquisition device 1, the information communication terminal 2, and the server 3, or may be realized by cooperation of the log acquisition device 1, the information communication terminal 2, and the server 3.

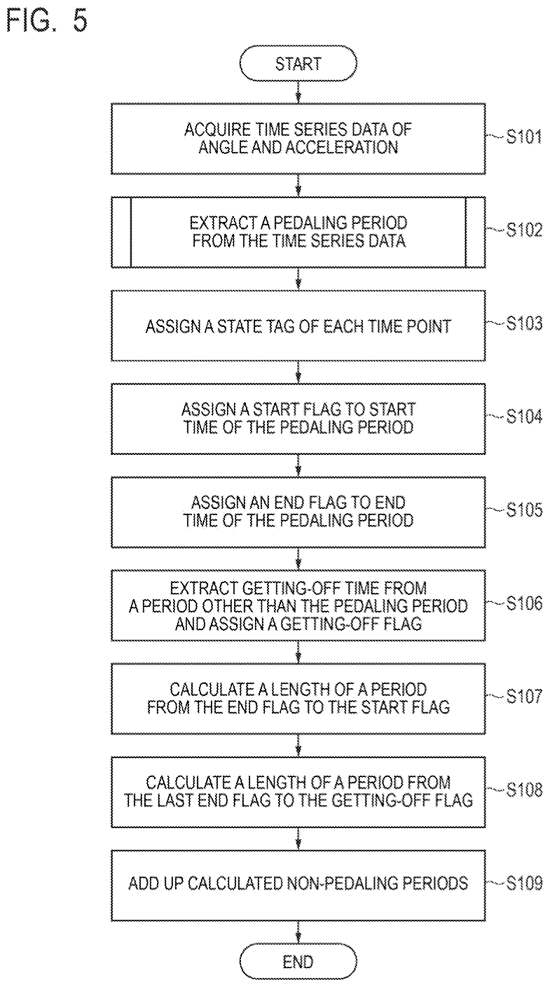

FIG. 5 is a flowchart illustrating an example of a log acquisition process performed by the log acquisition device 1 according to the present example embodiment. The process of FIG. 5 is performed at predetermined time intervals, for example. Alternatively, the process of FIG. 5 may be performed when the log acquisition device 1 detects that the user has got on the bicycle based on a change in acceleration or the like.

In step S101, the acquisition unit 120 controls the angular velocity sensor and the acceleration sensor of the IMU 12 to acquire time series data of angular velocity in three axial directions and acceleration in three directions. Thus, the acquisition unit 120 can acquire time changes in angular velocity and acceleration based on the motion of the foot of the user 4. The acquired time series data of angular velocity and acceleration is converted into digital data and then stored in the storage unit 160. These angular velocity and acceleration are referred to more generally as motion information. The motion information indicates a log of action of the user, and may be referred to as action information more generally.

The three directions of acceleration acquired by the acquisition unit 120 may be, for example, the width direction (left/right direction), the longitudinal direction (front/back direction), and the vertical direction of the foot of the user 4 provided with the IMU 12. These directions are referred to as x-axis, y-axis, and z-axis, respectively. The three axial directions of the angular velocity acquired by the acquisition unit 120 may be, for example, an adduction and an abduction of the foot about the z-axis (yaw), a pronation and a supination of the foot about the y-axis (pitch), and a bending and a stretching of the foot about x-axis (roll).

Here, in order to sufficiently acquire the feature included in the pedaling, it is desirable that the time series data of the angular velocity in the three axial directions and the acceleration in the three directions include data in a period corresponding to at least two pedaling cycles (rotation time corresponding to two cycles of the pedal). This is because the pedaling is a substantially periodic circular motion, and therefore, if at least two cycles can be extracted, it can be estimated that the same motion is repeated before and after the two cycles.

In step S102, the pedaling period extracting unit 130 extracts a pedaling period from time series data. Here, the pedaling period is a period during which the user 4 is in the pedaling state, that is, a period during which the user 4 is pedaling a bicycle.

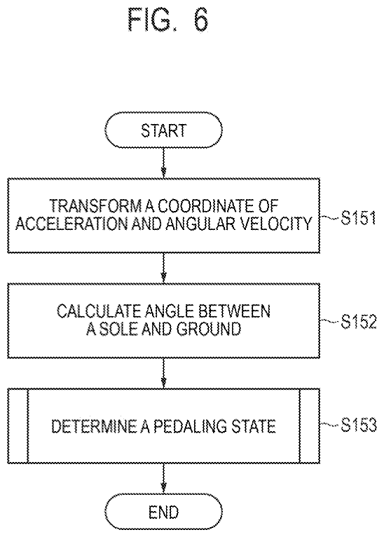

FIG. 6 is a flowchart illustrating an example of an extraction process of a pedaling period. The process of FIG. 6 is a subroutine corresponding to step S102 of FIG. 5.

In step S151, the coordinate system transforming unit 131 performs coordinate system transformation of angular velocity in three axial directions and acceleration in three directions. A coordinate system with respect to angular velocity and acceleration output by the IMU 12 is an inertial coordinate system. The coordinate system transforming unit 131 transforms the angular velocity and acceleration coordinate system into a coordinate system with respect to the foot of the user 4. Thus, the coordinate system of the angular velocity and the acceleration can be made suitable for calculating the angle between the sole and the ground. The transformation of the coordinate system is realized, for example, by multiplying the base vector of the inertial coordinate system by the direction cosine matrix E using the Euler angle and rotating the base vector.

An example of transformation of the coordinate system by the direction cosine matrix E is described more specifically. In a case where the base vector of the inertial coordinate system is [xᵢ, yᵢ, zᵢ], and the base vector of the coordinate system with respect to the foot is [x_{b}, y_{b}, z_{b}], a conversion formula between them is expressed by the following equation (1).

$$\begin{matrix}[ Math . 1 ] & \\ [ \begin{matrix}{x}_{b} \\ {y}_{b} \\ {z}_{b}\end{matrix} ] = [ \begin{matrix}{E}_{11} & {E}_{12} & {E}_{13} \\ {E}_{21} & {E}_{22} & {E}_{23} \\ {E}_{31} & {E}_{32} & {E}_{33}\end{matrix} ] [ \begin{matrix}{x}_{i} \\ {y}_{i} \\ {z}_{i}\end{matrix} ] = E [ \begin{matrix}{x}_{i} \\ {y}_{i} \\ {z}_{i}\end{matrix} ] & ( 1 )\end{matrix}$$

In a case where angle acquired by rotating the base vector of the inertial coordinate system by angles of ψ (psi), θ (theta), and σ (phi) in the order of z, y, and x is an Euler angle of the coordinate system transformation, the direction cosine matrix E is expressed by the following equation (2).

$$\begin{matrix}[ Math . 2 ] & \\ E = [ \begin{matrix}1 & 0 & 0 \\ 0 & cos ϕ & sin ϕ \\ 0 & - s in ϕ & cos ϕ\end{matrix} ] [ \begin{matrix}cos θ & 0 & - s in θ \\ 0 & 1 & 0 \\ sin θ & 0 & cos θ\end{matrix} ] [ \begin{matrix}cos ψ & sin ψ & 0 \\ - s in ψ & cos ψ & 0 \\ 0 & 0 & 1\end{matrix} ] = [ \begin{matrix}cos θ cos ψ & cos θ sin ψ & - s in θ \\ sin ϕ sin θ cos ψ - cos ϕ sin ψ & sin ϕ sin θ sin ψ + cos ϕ cos ψ & sin ϕ cos θ \\ cos ϕ sin θ cos ψ + sin ϕ sin ψ & cos ϕ sin θ sin ψ - sin ϕ cos ψ & cos ϕ cos θ\end{matrix} ] & ( 2 )\end{matrix}$$

Note that the calculation method used for conversion of the coordinate system is merely an example, and other calculation methods may be used. For example, a calculation method using a quaternion may be applied.

In step S152, the angle calculation unit 132 calculates the angle between the sole of the user 4 and the ground from the angular velocity in the three axial directions and the acceleration in the three directions after being transformed into the coordinate system with respect to the foot of the user 4. As a specific example of this process, there is a method in which angular velocity in three axial directions and acceleration in three directions are input to a Madgwick filter (Non Patent Literature 1), and a rotation angle in three axial directions of the foot is output. The rotation angles in the three axial directions acquired by the Madgwick filter are the angles of adduction or abduction of the foot, the angle of pronation or supination of the foot, and the angle of bending or stretching of the foot. Of these three angles, the angle of stretching or bending of the foot corresponds to the angle between the sole of the foot of the user 4 and the ground.

In step S153, the pedaling period extracting unit 130 performs a pedaling state determination process for determining whether or not the user 4 is in the pedaling state in which the user 4 is pedaling the bicycle, based on at least the above-described angle.

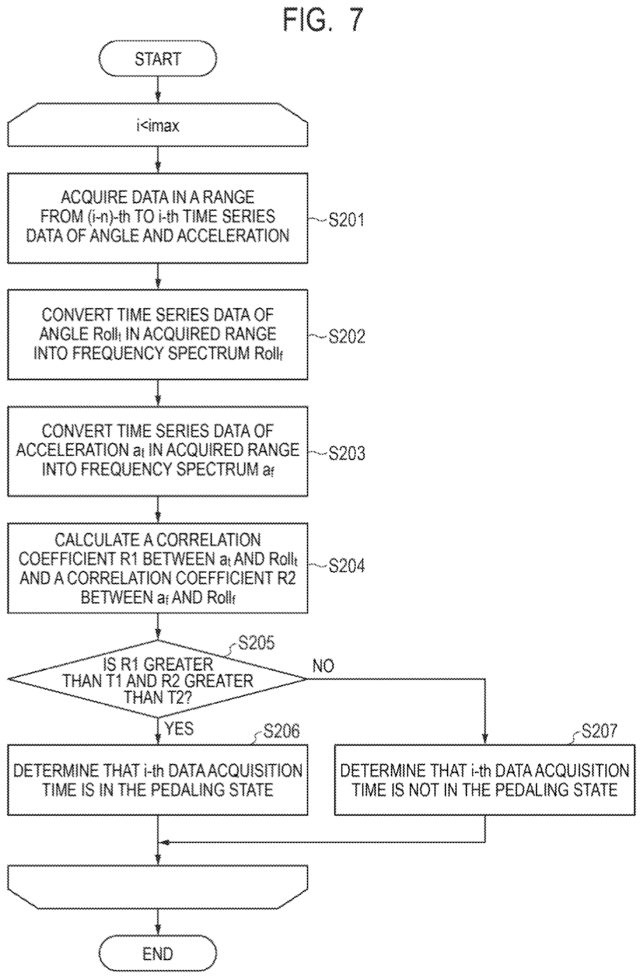

FIG. 7 is a flowchart illustrating an example of a pedaling state determination process. The process of FIG. 7 is a subroutine corresponding to step S153 of FIG. 6. This process is a loop process in which steps S201 through S207 are repeated for each data. In FIG. 7, i represents a data number of time series data of the input angle and acceleration. The process from steps S201 through step S207 are repeated until the data number reaches the predetermined upper limit value imax from the initial value.

In step S201, the data selecting unit 133 acquires data in the range from the (i-n)-th to the i-th of the time series data of the angle and the time series data of the acceleration. This process is for specifying a time range of time series data used for conversion into a frequency domain in step S202 and step S203 described later. Therefore, the process of the data selecting unit 133 corresponds to a process of multiplying the time series data by a rectangular window having a width n. Note that the process may be modified to use another window function, and for example, a Gaussian window, a Hanning window, or the like may be applied.

In step S202, the data conversion unit 134 converts the time series data of the angle Rollₜ in the range acquired in step S201 into the frequency spectrum Roll_{f}. This process may be any process as long as it can convert time domain data into frequency domain data, and may be Fourier transform, for example. The algorithm used for the Fourier transform may be, for example, a fast Fourier transform.

In step S203, as in step S202, the data conversion unit 134 converts the time series data of the acceleration at in the range acquired in step S201 into the frequency spectrum a_{f}.

In step S204, the similarity degree calculation unit 135 calculates a correlation coefficient R1 between time series data of the acceleration aₜ and time series data of the angle Rollₜ. Further, the similarity degree calculation unit 135 calculates a correlation coefficient R2 between the frequency spectrum of the acceleration of and the frequency spectrum of the angle Roll_{f}. Note that the correlation coefficients R1 and R2 may typically be Pearson's product moment correlation coefficients. The correlation coefficients R1 and R2 may be referred to as a first similarity degree and a second similarity degree, respectively.

In step S205, the comparison unit 136 compares the correlation coefficients R1 and R2 with predetermined threshold values T1 and T2. When the correlation coefficient R1 is greater than the threshold value T1 and the correlation coefficient R2 is greater than the threshold value T2 (YES in step S205), the process proceeds to step S206. If the above condition is not satisfied (NO in step S205), the process proceeds to step S207. The threshold values T1 and T2 may be more generally referred to as a first threshold value and a second threshold value, respectively.

In step S206, the pedaling period extracting unit 130 determines that the user 4 pedaled the bicycle at the i-th data acquisition time (that is, the user 4 was in the pedaling state). The determination result is stored in the storage unit 160 in association with the data number i or the time corresponding thereto.

In step S207, the pedaling period extracting unit 130 determines that the user 4 did not pedal the bicycle at the i-th data acquisition time (that is, the user 4 was not in the pedaling state). The determination result is stored in the storage unit 160 in association with the data number i or the time corresponding thereto.

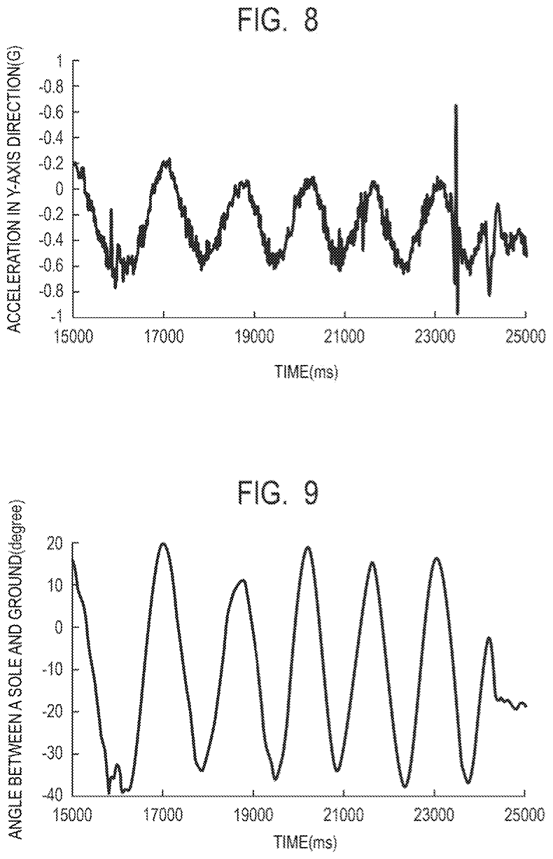

In the above-described pedaling state determination process, the angle between the sole of the foot and the ground is used for determination. The reason why it is possible to accurately determine whether or not the user 4 is pedaling is described. FIG. 8 is a graph illustrating an example of time series data of acceleration in a pedaling state. The horizontal axis of FIG. 8 represents time in units of milliseconds (ms), and the vertical axis of FIG. 8 represents acceleration in the y-axis direction, that is, in the longitudinal direction of the foot. The unit G of the vertical axis is a unit of acceleration based on the standard gravitational acceleration (about 9.8 m/s²). When the user 4 is pedaling, the foot of the user 4 is rotating, so that the acceleration has a waveform close to a sine wave. As can be understood from FIG. 8, the acceleration includes large noise due to various factors such as vibration of the bicycle. In some cases, as in the vicinity of 23500 ms in FIG. 8, a large noise exceeding the amplitude of the sine wave may be generated, and if the determination of the pedaling state is performed using only the acceleration, such noise may affect the determination accuracy.

FIG. 9 is a graph illustrating an example of time series data of angle between a sole and ground in a pedaling state. The horizontal axis of FIG. 9 represents time, and the vertical axis of FIG. 9 represents the angle between the sole of the foot and the ground. As can be understood from FIG. 9, the noise included in the angle is smaller than the noise included in the acceleration. Therefore, determination accuracy can be improved by performing determination of the pedaling state using an algorithm utilizing the angle.

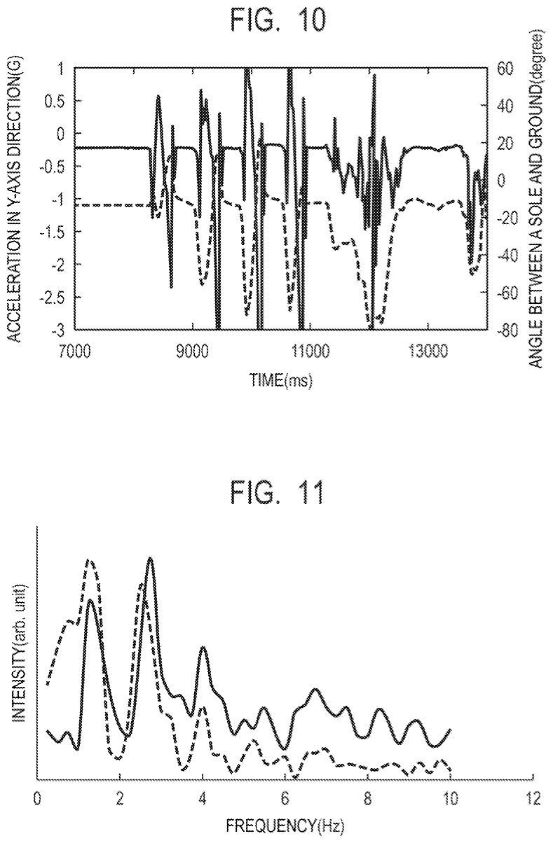

In the above-described pedaling state determination process, the determination is performed using the correlation coefficient between the acceleration and the angle. The reason why whether or not the user 4 is pedaling can be determined with higher accuracy is described. First, waveforms of acceleration and angle when the user 4 is walking is described with reference to FIG. 10 and FIG. 11 as an example of a case where the user 4 is not pedaling (non-pedaling state). FIG. 10 is a graph illustrating an example of time series data of acceleration and time series data of angle when the user 4 is walking. The horizontal axis of FIG. 10 represents time, the left axis of FIG. 10 represents acceleration in the y-axis direction, and the right axis of FIG. 10 represents angle between the sole of the foot and the ground. The solid line graph of FIG. 10 indicates the acceleration of the left axis, and the broken line graph of FIG. 10 indicates the angle of the right axis.

FIG. 11 is a graph illustrating an example of a frequency spectrum of acceleration and a frequency spectrum of an angle when the user 4 is walking. The horizontal axis of FIG. 11 represents the frequency in units of Hertz (Hz), and the vertical axis of FIG. 11 represents the intensity in arbitrary units. The solid line graph of FIG. 11 represents the frequency spectrum of the acceleration, and the broken line graph of FIG. 11 represents the frequency spectrum of the angle.

As can be understood from FIG. 10 and FIG. 11, when the user 4 walks, the waveform of the acceleration and the waveform of the angle are not similar to each other in both the time series data and the frequency spectrum. Therefore, when the user 4 walks, the correlation coefficient between the acceleration and the angle is a small value.

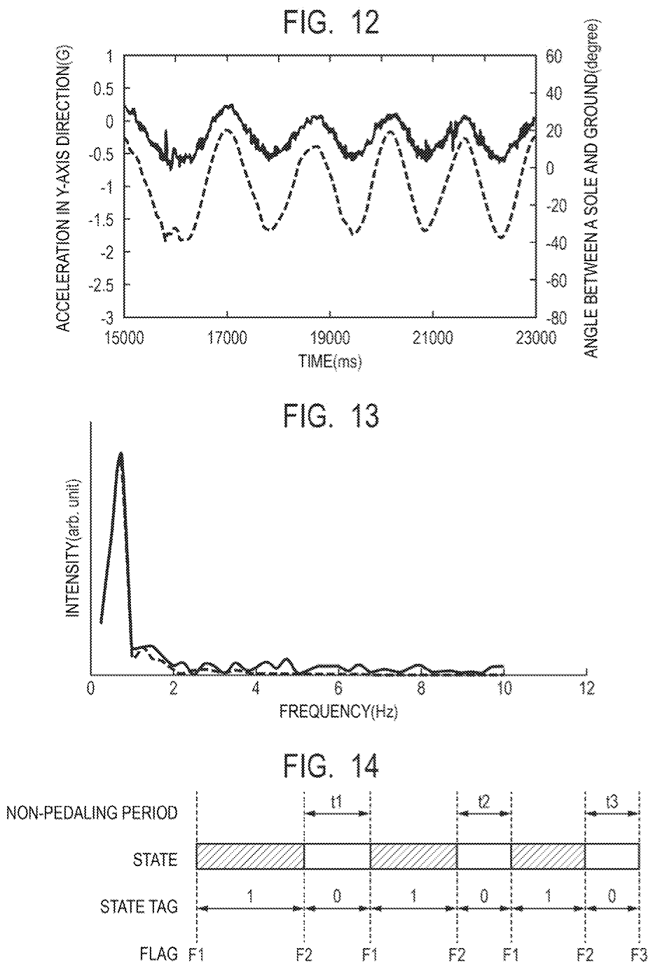

Next, waveforms of acceleration and angle when the user 4 is pedaling (pedaling state) is described with reference to FIG. 12 and FIG. 13. The notations of the graphs are the same as those in FIG. 10 and FIG. 11, and therefore the description thereof is omitted. As can be understood from FIG. 12 and FIG. 13, in both the time series data and the frequency spectrum, the waveform of the acceleration and the waveform of angle are similar to each other. Therefore, in the pedaling state, the correlation coefficient between the acceleration and the angle is greater than that in the walking state.

As described above, in the pedaling state, the similarity degree between the acceleration and the angle is high and the correlation coefficient is large as compared with the non-pedaling state. Therefore, the correlation coefficient is calculated as an index of the similarity degree between the acceleration and the angle, and the magnitude relation between the correlation coefficient and the threshold value is used as the determination condition, whereby it is possible to determine the pedaling state with higher accuracy. An index other than the correlation coefficient may be used as long as the determination method uses the similarity degree between the acceleration and the angle. For example, covariance may be used as a determination condition.

Further, in this determination, by referring to both the time series data, which is the waveform in the time domain, and the frequency spectrum, which is the waveform in the frequency domain, it is possible to more reliably determine the pedaling state. However, the determination may be performed using only the time series data or using only the frequency spectrum. In this case, the process is simplified, and the amount of calculation can be reduced.

As described above, the state of the user 4 riding the bicycle can be accurately determined by determining whether or not the user 4 is in the pedaling state based on the angle between the sole of the foot and the ground.

Referring back to FIG. 5, a process after pedaling period extraction in step S102 is described. In step S103, the identifier assigning unit 140 assigns a state tag at each time point to the time series data in which extraction of the pedaling period has been completed. In step S104, the identifier assigning unit 140 assigns a start flag to the start time of the pedaling period. In step S105, the identifier assigning unit 140 assigns an end flag to the end time of the pedaling period. In step S106, the identifier assigning unit 140 extracts getting-off time at which the user 4 gets off the bicycle from a period other than the pedaling period, and assigns a getting-off flag to the getting-off time.

The assignment of the identifiers (the state flag, the start flag, the end flag, and the getting-off flag) from step S103 to step S106 is described in more detail. FIG. 14 is a diagram illustrating an example of a relation between a pedaling state and an identifier.

“state” in FIG. 14 indicates whether or not the user 4 is in the pedaling state. A hatched frame in the “state” indicates a pedaling period, and a non-hatched frame indicates a non-pedaling period. The horizontal direction of FIG. 14 indicates the elapsed time. That is, it can be seen from FIG. 14 that the pedaling period and the non-pedaling period are alternately repeated. The non-pedaling period in this case is a period during which the bicycle is inertially traveling while the user 4 temporarily stops pedaling.

The “state tag” in FIG. 14 indicates the value of the state tag assigned in step S103. The state tag is an identifier that indicates whether or not the user 4 is in the pedaling state in a certain range of time. In FIG. 14, the state tag of the pedaling state is “1”, and the state tag of the non-pedaling state is “0”, but other identifiers may be used. The identifier assigning unit 140 assigns a state tag based on the extraction result of the pedaling period in step S102.

“flag” in FIG. 14 indicates a kind of flag assigned in steps S104 to S106. The flag is an identifier indicating a change in state at certain time. In FIG. 14, the start flag indicating the start time of the pedaling period is “F1”, the end flag indicating the end time of the pedaling period is “F2”, and the getting-off flag indicating the getting-off time is “F3”, but other identifiers may be used.

The method of setting the start flag in step S104 may be, for example, detecting time when the value of the state tag changes from 0 to 1 and setting the start flag at that time. The method of setting the end flag in step S105 may be, for example, detecting a time when the value of the state tag changes from 1 to 0 and setting the end flag at that time.

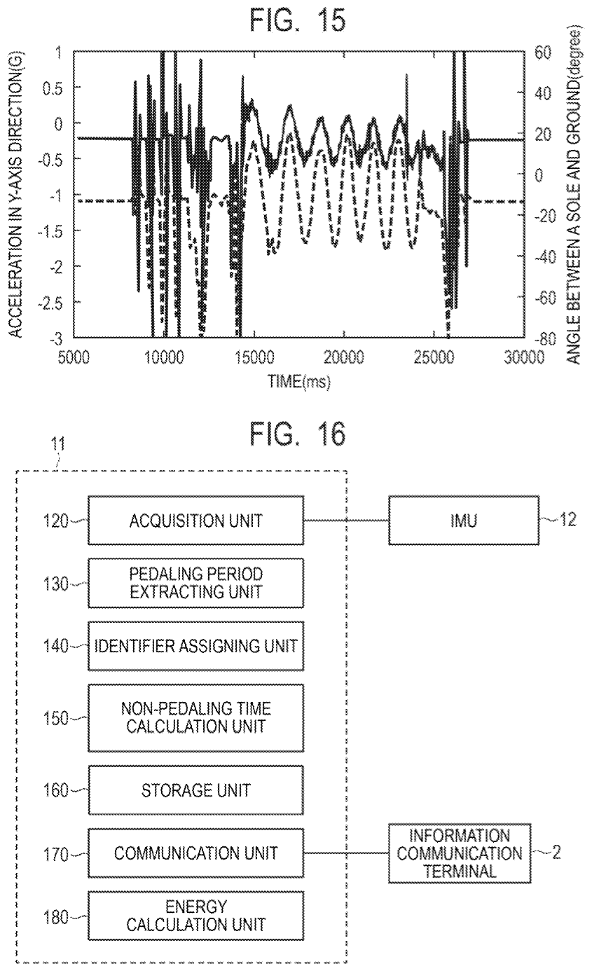

An example of a method of setting the getting-off flag in step S106 is described. FIG. 15 is a graph illustrating an example of time series data of acceleration and time series data of angle in the vicinity of the getting-off time. The notation of the graph is the same as that in FIG. 10, and the description thereof is omitted. A period from around 15000 ms to around 24000 ms is a pedaling state, and a period after 24000 ms is a non-pedaling state. In the vicinity of 26000 ms, large a fluctuation in acceleration and angle is observed. This fluctuation is due to the motion of the foot when the user 4 is getting off the bicycle. Therefore, by determining whether or not the level of the acceleration or the angle exceeds a predetermined threshold value after the end of the last pedaling period of the plurality of pedaling periods, it is possible to determine that the user 4 has gotten off a vehicle. In addition, by acquiring the time at which the getting-off is detected, the getting-off time can be acquired, and the getting-off flag can be set at that time. A specific example of the threshold value of the acceleration may be, for example, 2G. A specific example of the threshold value of the angle may be, for example, 40°.

Referring back to FIG. 5, the process after the assignment of the identifier from step S104 to step S106 is described. In step S107, the non-pedaling time calculation unit 150 calculates the length of a period from a certain end flag to the next start flag. In this way, in a case where two consecutive pedaling periods are referred to as a first pedaling period and a second pedaling period, respectively, the length of the non-pedaling period from the end time of the first pedaling period to the start time of the second pedaling period is calculated. In the example of FIG. 14, the period t1 and the period t2 illustrated in the “non-pedaling period” correspond to the non-pedaling period calculated in the process of step S107.

In step S108, the non-pedaling time calculation unit 150 calculates the length of the period from the end flag to the getting-off flag. In the example of FIG. 14, the period t3 indicated by the “non-pedaling period” corresponds to the non-pedaling period calculated in the process of step S108.

In step S109, the non-pedaling time calculation unit 150 adds the non-pedaling period calculated in step S107 and the non-pedaling period calculated in step S108. Thus, the total value of the length of the non-pedaling periods (non-pedaling time) from when the user 4 gets on the bicycle to when the user 4 gets off the bicycle can be acquired. In the example of FIG. 14, this process corresponds to addition process of t1+t2+t3. The calculation result is stored in the storage unit 160.

According to the present example embodiment, it is possible to extract the pedaling period, calculate the non-pedaling time based on the start time and the end time of the pedaling period, and calculate the time during which the user 4 is not pedaling in the riding time of the bicycle. Thus, the information processing device capable of more appropriately determining the state of the user 4 riding the bicycle is provided.

In the method of calculating the non-pedaling time in the present example embodiment, a method of individually calculating and adding the non-pedaling time is exemplified, but other methods may be applied. For example, the non-pedaling time can be calculated in the same manner even when the length of the pedaling period (that is, the length of the period from a certain start flag to the next end flag) is added and subtracted from the entire riding time of the bicycle.

### SECOND EXAMPLE EMBODIMENT

The energy calculation system of the present example embodiment is an example of utilizing the function of determining the pedaling state by the log acquisition system of the first example embodiment. There is a need to acquire a log of daily energy consumption (so-called consumed calories). As a part of health management, the energy calculation system is a system that can meet the above needs by calculating the energy consumed by the user 4 when the user 4 rides the bicycle. Description of portions common to those in the first example embodiment is omitted.

FIG. 16 is a functional block diagram of the information processing device 11 included in the energy calculation system according to the present example embodiment. The energy calculation system of the present example embodiment is acquired by adding an energy calculation unit 180 to the information processing device 11 of the log acquisition system of the first example embodiment. The CPU 111 realizes the function of the energy calculation unit 180 by loading a program stored in the ROM 113, the flash memory 114, or the like into the RAM 112 and executing the program. In FIG. 16, the energy calculation unit 180 is provided in the information processing device 11, but this function may be provided in the information communication terminal 2 or the server 3.

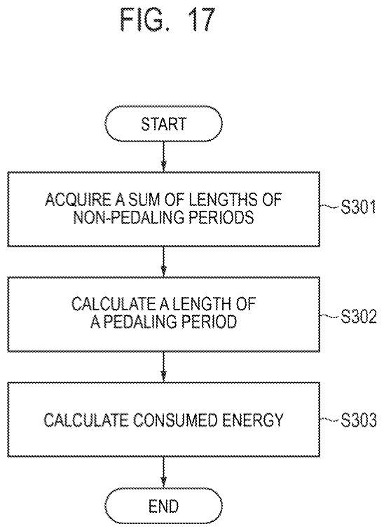

FIG. 17 is a flowchart illustrating an example of an energy calculation process performed by the energy calculation unit 180 according to the present example embodiment. The process of FIG. 17 is performed, for example, after the end of the process according to the flowchart of FIG. 5. Alternatively, the process of FIG. 17 may be performed based on an operation of energy calculation by the user 4.

In step S301, the energy calculation unit 180 acquires the sum of the lengths of the non-pedaling periods from the storage unit 160. In step S302, the energy calculation unit 180 calculates the length of the pedaling period by subtracting the sum of the lengths of the non-pedaling periods from the time when the user 4 was riding the bicycle.

Note that the process of step S301 and step S302 may be a process adding up the periods in which the user is in the pedaling state (pedaling period) by acquiring the determination result of the pedaling state corresponding to each data acquisition time, and calculating the length of the pedaling period.

In step S303, the energy calculation unit 180 calculates the energy consumed by the user 4 due to the riding of the bicycle by the user 4, based on the length of the pedaling period. For example, the following equation (3) can be used as a calculation equation used for this calculation.

Consumed energy=exercise intensity (METs)×length of pedaling period×body weight×coefficient (3)

In Equation (3), METs, which is a unit of exercise intensity, represents how many times the energy consumption is as compared with the rest state during exercise. Depending on the speed, the inclination of the riding route, and the like, the METs of the bicycle riding is, for example, 4.0 (METs) or 6.8 (METs). The value of the exercise intensity may be input by the user 4 in advance with reference to a METs table or the like, or may be automatically set based on the speed of the bicycle or the like calculated from the acceleration acquired by the IMU 12. In Equation (3), the coefficient is about 1.05 when the unit of the length of the pedaling period is time (hour), the unit of the body weight is kg, and the unit of the consumed energy is kcal.

In the pedaling state, by pedaling, the energy consumption is increased as compared with the case of the non-pedaling state. By focusing attention on the length of the pedaling period, the energy calculation unit 180 of the present example embodiment can calculate the consumed energy more accurately than the case where the consumed energy is calculated based only on the length of time during which the user 4 is on the bicycle.

The energy calculation system of the present example embodiment uses the information processing device 11 that can determine the state of the user 4 riding the bicycle more appropriately. Thus, an energy calculation system capable of accurately calculating consumed energy is provided.

### THIRD EXAMPLE EMBODIMENT

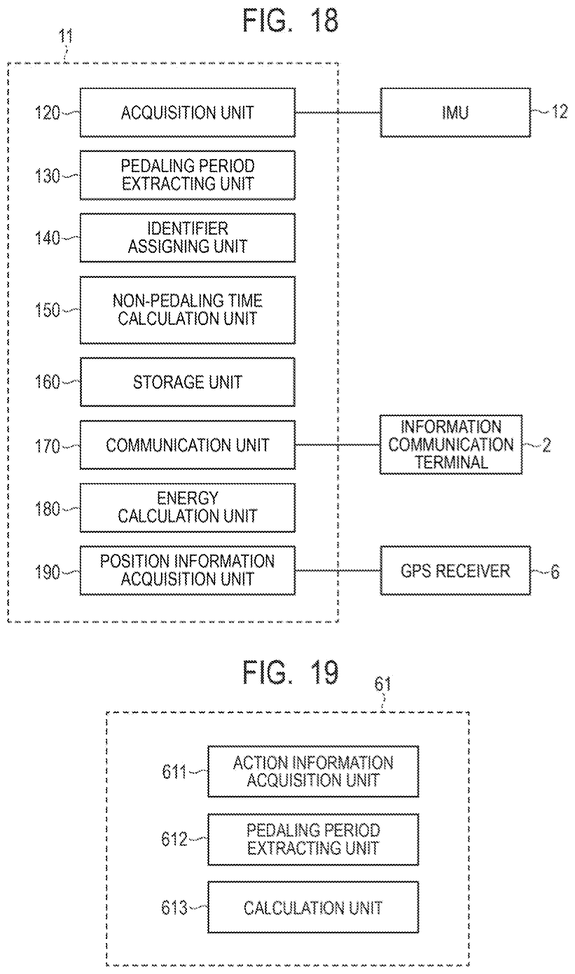

The energy calculation system of the present example embodiment is a modified example of the energy calculation system of the second example embodiment. FIG. 18 is a functional block diagram of the information processing device 11 included in the energy calculation system according to the present example embodiment. The energy calculation system of the present example embodiment is acquired by adding the global positioning system (GPS) receiver 6 and the position information acquisition unit 190 to the energy calculation system of the second example embodiment. Description of portions common to those in the second example embodiment is omitted.

The GPS receiver 6 acquires signals from a plurality of GPS satellites. The GPS receiver 6 may be provided in the log acquisition device 1 or may be provided in the information communication terminal 2.

The position information acquisition unit 190 is provided in the information processing device 11. The position information acquisition unit 190 acquires the position information of the user 4 based on the plurality of signals acquired by the GPS receiver 6. The CPU 111 realizes the function of the position information acquisition unit 190 by loading a program stored in the ROM 113, the flash memory 114, or the like into the RAM 112 and executing the program. The process of calculating the position information from the signals acquired from the GPS satellites may be performed in the GPS receiver 6.

The energy calculation system of the present example embodiment can further acquire the position information of the user 4 in addition to the consumed energy. The position information may be used as one of logs for various applications. For example, when the change in position in the period during which the user 4 is riding the bicycle is small or the speed is small, it is assumed that the user 4 is not riding the bicycle on the road but performing training by a stationary bicycle. Therefore, whether or not the bicycle is the stationary bicycle is determined based on the position information, and this information is recorded as a log of action, whereby the log can be made more reliable. Further, when the exercise intensity (METs) differs between the training in the stationary bicycle and the riding of the bicycle on the road, the consumption energy can be calculated more accurately by considering the exercise intensity.

Although the GPS receiver 6 is used as an example of the method of acquiring the position information, the position information may be acquired by other methods. For example, the GPS receiver 6 may be replaced with one that receives signals from satellites other than GPS satellites. Examples thereof include global navigation satellite system (GLONASS), Galileo, Beidou navigation satellite system (BDS), and the like. Alternatively, the position information may be acquired based on the position of the access point communicatively connected by Wi-Fi. Alternatively, the position information may be acquired by integrating the acceleration acquired by the IMU 12.

The device or system described in the above example embodiments can also be configured as in the following fourth example embodiment.

### FOURTH EXAMPLE EMBODIMENT

FIG. 19 is a functional block diagram of the information processing device 61 according to the fourth example embodiment. The information processing device 61 includes an action information acquisition unit 611, a pedaling period extracting unit 612, and a calculation unit 613. The action information acquisition unit 611 acquires action information of a user. The pedaling period extracting unit 612 extracts a plurality of pedaling periods during which the user pedals a bicycle from the action information. The calculation unit 613 calculates a non-pedaling time during which the user is on the bicycle and does not pedal based on a start time of each of the plurality of pedaling periods and an end time of each of the plurality of pedaling periods.

According to the present example embodiment, the information processing device 61 capable of more appropriately determining the state of the user riding the bicycle is provided.

### MODIFIED EXAMPLE EMBODIMENTS

The present invention is not limited to the example embodiments described above, and may be suitably modified within the scope of the present invention. For example, an example in which a part of the configuration of one example embodiment is added to another example embodiment or an example in which a part of the configuration of one example embodiment is replaced with another example embodiment is also an example embodiment of the present invention.

In the above-described example embodiments, the motion measurement device including the angular velocity sensor that measures the angular velocity in the three axial directions and the acceleration sensor that measures the acceleration in the three directions is used, but sensors other than these may also be used. For example, a magnetic sensor that detects geomagnetism by detecting magnetism in three directions to identify an azimuth may be further used. Even in this case, the same processing as the above-described example embodiments can be applied, and the accuracy can be further improved.

Although the log acquisition process is performed in the log acquisition device 1 in the above-described example embodiments, this function may be provided in the information communication terminal 2. In this case, the information communication terminal 2 functions as a log acquisition device.

In the above-described example embodiment, the pedaling period is extracted based on the motion information acquired by the IMU 12, but this is merely an example, and the pedaling period may be extracted by other methods. For example, a rotation sensor for detecting the rotation of the pedal is provided in the bicycle, and the time series data of the output of the rotation sensor is acquired as the action information, whereby the pedaling period can be extracted in the same manner as in the above-described example embodiments.

A processing method in which a program for operating the configuration of the above-described example embodiments are recorded in a storage medium so as to implement the functions of the above-described example embodiments, the program recorded in the storage medium is read as code, and the program is executed in a computer is also included in the scope of each example embodiment. That is, a computer-readable storage medium is also included in the scope of the example embodiments. Further, not only the storage medium in which the above program is recorded, but also the program itself is included in each example embodiment. In addition, one or more components included in the above-described example embodiments may be a circuit such as an application specific integrated circuit (ASIC), a field programmable gate array (FPGA) configured to implement the functions of each component.

As the storage medium, for example, a floppy (registered trademark) disk, a hard disk, an optical disk, a magneto-optical disk, a compact disk (CD)-ROM, a magnetic tape, a nonvolatile memory card, or a ROM can be used. Further, the scope of each example embodiment is not limited to the case where the processing is executed by the program alone recorded in the storage medium, and a case where the processing is executed by operating on an operating system (OS) n cooperation with the functions of other software and extension board is also included in the scope of each example embodiment.

The service realized by the functions of the above-described example embodiments may be provided to the user in the form of a software as a service (SaaS).

It should be noted that the above-described example embodiments are merely examples of embodying the present invention, and the technical scope of the present invention should not be limitedly interpreted by these. That is, the present invention can be implemented in various forms without departing from the technical idea or the main features thereof.

The whole or part of the example embodiments disclosed above can be described as, but not limited to, the following supplementary notes.

### (Supplementary Note 1)

An information processing device comprising:

an action information acquisition unit configured to acquire action information of a user;

a pedaling period extracting unit configured to extract a plurality of pedaling periods during which the user pedals a bicycle from the action information; and

a calculation unit configured to calculate a non-pedaling time during which the user is on the bicycle and does not pedal based on a start time of each of the plurality of pedaling periods and an end time of each of the plurality of pedaling periods.

### (Supplementary Note 2)

The information processing device according to supplementary note 1, wherein the calculation unit calculates a length of a period from the end time of a first pedaling period of the plurality of pedaling periods to the start time of a second pedaling period next to the first pedaling period of the plurality of pedaling periods as the non-pedaling time.

### (Supplementary Note 3)

The information processing device according to supplementary 1 or 2, wherein the calculation unit calculates the non-pedaling time further based on a getting-off time at which the user gets off the bicycle.

### (Supplementary Note 4)

The information processing device according to supplementary note 3,

wherein the action information includes time series data of motion information of a foot of the user, and

wherein the calculation unit extracts a time at which a level of the motion information exceeds a threshold value after the end time of a last pedaling period of the plurality of pedaling periods as the getting-off time.

### (Supplementary Note 5)

The information processing device according to supplementary note 3 or 4, wherein the calculation unit calculates a length of a period from the end time of a last pedaling period of the plurality of pedaling periods to the getting-off time as the non-pedaling time.

### (Supplementary Note 6)

The information processing device according to any one of supplementary notes 1 to 5, wherein the calculation unit sums up a plurality of the non-pedaling time.

### (Supplementary Note 7)

The information processing device according to any one of supplementary notes 1 to 6 further comprising a position information acquisition unit configured to acquire position information of the user.

### (Supplementary Note 8)

The information processing device according to supplementary note 7, wherein the position information is used to determine whether or not the bicycle is stationary.

### (Supplementary Note 9)

The information processing device according to any one of supplementary notes 1 to 8,

wherein the action information includes the motion information of the foot of the user measured by a motion measurement device; and

wherein the pedaling period extracting unit determines whether or not the user is in a pedaling state in which the user pedals based on an angle between a sole and a ground generated from the motion information.

### (Supplementary Note 10)

The information processing device according to supplementary note 9, wherein the motion information includes acceleration of the foot.

### (Supplementary Note 11)

The information processing device according to supplementary note 10, wherein the pedaling period extracting unit determines whether or not the user is in the pedaling state further based on the acceleration.

### (Supplementary Note 12)

The information processing device according to supplementary note 11, wherein the pedaling period extracting unit determines whether or not the user is in the pedaling state based on time series data of the angle and time series data of the acceleration.

### (Supplementary Note 13)

The information processing device according to supplementary note 12, wherein the pedaling period extracting unit determines whether or not the user is in the pedaling state based on a first similarity degree between the time series data of the angle and the time series data of the acceleration.

### (Supplementary Note 14)

The information processing device according to supplementary note 13, wherein the first similarity degree includes a correlation coefficient between the time series data of the angle and the time series data of the acceleration.

### (Supplementary Note 15)

The information processing device according to any one of supplementary notes 12 to 14, wherein the pedaling period extracting unit determines whether or not the user is in the pedaling state further based on a frequency spectrum of the angle and a frequency spectrum of the acceleration acquired by transforming the time series data of the angle and the time series data of the acceleration into a frequency domain.

### (Supplementary Note 16)

The information processing device according to supplementary note 15, wherein the pedaling period extracting unit determines whether or not the user is in the pedaling state based on a second similarity degree between the frequency spectrum of the angle and the frequency spectrum of the acceleration.

### (Supplementary Note 17)

The information processing device according to supplementary note 16, wherein the second similarity degree includes a correlation coefficient between the frequency spectrum of the angle and the frequency spectrum of the acceleration.

### (Supplementary Note 18)

The information processing device according to supplementary note 16 or 17, wherein the pedaling period extracting unit determines the user is in the pedaling state in a case where a first similarity degree between the time series of the angle and the time series data of the acceleration is greater than a first threshold value and a second similarity degree between the frequency spectrum of the angle and the frequency spectrum of the acceleration is greater than a second threshold value.

### (Supplementary Note 19)

The information processing device according to any one of supplementary notes 12 to 18, wherein the time series data includes at least two periods of pedaling cycles.

### (Supplementary Note 20)

The information processing device according to any one of supplementary notes 10 to 19, wherein the motion information further includes angular velocity of the foot.

### (Supplementary Note 21)

The information processing device according to supplementary note 20, wherein the pedaling period extracting unit transforms a coordinate system of the acceleration and the angular velocity included in the motion information into a coordinate system with respect to the foot.

### (Supplementary Note 22)

The information processing device according to supplementary note 20 or 21, wherein the pedaling period extracting unit calculates the angle using the acceleration and the angular velocity.

### (Supplementary Note 23)

The information processing device according to supplementary note 22, wherein the pedaling period extracting unit calculates the angle using a Madgwick filter.

### (Supplementary Note 24)

The information processing device according to any one of supplementary notes 9 to 23, wherein the motion measurement device is provided at a position corresponding to an arch of the foot.

### (Supplementary Note 25)

A log acquisition system comprising: the information processing device according to any one of supplementary notes 9 to 24; and the motion measurement device.

### (Supplementary Note 26)

An energy calculation system comprising an energy calculation unit configured to calculate energy consumed by the user by riding the bicycle based on a length of the pedaling period or the non-pedaling time acquired by the information processing device according to any one of supplementary notes 1 to 24.

### (Supplementary Note 27)

An information processing method comprising:

acquiring action information of a user;

extracting a plurality of pedaling periods during which the user pedals a bicycle from the action information; and

calculating a non-pedaling time during which the user is on the bicycle and does not pedal based on a start time of each of the plurality of pedaling periods and an end time of each of the plurality of pedaling periods.

### (Supplementary Note 28)

A storage medium storing a program that causes a computer to perform:

acquiring action information of a user;

extracting a plurality of pedaling periods during which the user pedals a bicycle from the action information; and

calculating a non-pedaling time during which the user is on the bicycle and does not pedal based on a start time of each of the plurality of pedaling periods and an end time of each of the plurality of pedaling periods.

### REFERENCE SIGNS LIST

1 log acquisition device
2 information communication terminal
3 server
4 user
5 shoe
6 GPS receiver
11, 61 information processing device
12 IMU
13 battery
111, 201 CPU
112, 202 RAM
113, 203 ROM
114, 204 flash memory
115, 205 communication I/F
116 IMU control device
120 acquisition unit
130, 612 pedaling period extracting unit
131 coordinate system transforming unit
132 angle calculation unit
133 data selecting unit
134 data conversion unit
135 similarity degree calculation unit
136 comparison unit
140 identifier assigning unit
150 non-pedaling time calculation unit
160 storage unit
170 communication unit
180 energy calculation unit
190 position information acquisition unit
206 input device
207 output device
611 action information acquisition unit

What is claimed is:

1. An information processing device comprising:
a memory configured to store instructions; and
a processor configured to execute the instructions to:
acquire action information of a user;
extract a plurality of pedaling periods during which the user pedals a bicycle from the action information; and
calculate a non-pedaling time during which the user is on the bicycle and does not pedal based on a start time of each of the plurality of pedaling periods and an end time of each of the plurality of pedaling periods.

2. The information processing device according to claim 1, wherein a length of a period from the end time of a first pedaling period of the plurality of pedaling periods to the start time of a second pedaling period next to the first pedaling period of the plurality of pedaling periods is calculated as the non-pedaling time.

3. The information processing device according to claim 1, wherein the non-pedaling time is calculated further based on a getting-off time at which the user gets off the bicycle.

4. The information processing device according to claim 3,
wherein the action information includes time series data of motion information of a foot of the user, and
wherein a time at which a level of the motion information exceeds a threshold value after the end time of a last pedaling period of the plurality of pedaling periods is extracted as the getting-off time.

5. The information processing device according to claim 3, wherein a length of a period from the end time of a last pedaling period of the plurality of pedaling periods to the getting-off time is calculated as the non-pedaling time.

6. The information processing device according to claim 1, wherein a plurality of the non-pedaling time is summed up.

7. The information processing device according to claim 1, wherein the processor is further configured to execute the instructions to acquire position information of the user.

8. The information processing device according to claim 7, wherein the position information is used to determine whether or not the bicycle is stationary.

9. The information processing device according to claim 1,
wherein the action information includes the motion information of the foot of the user measured by a motion measurement device; and
wherein the processor is further configured to execute the instructions to determine whether or not the user is in a pedaling state in which the user pedals based on an angle between a sole and a ground generated from the motion information.

10. The information processing device according to claim 9, wherein the motion information includes acceleration of the foot.

11. The information processing device according to claim 10, wherein whether or not the user is in the pedaling state is determined further based on the acceleration.

12. The information processing device according to claim 11, wherein whether or not the user is in the pedaling state is determined based on time series data of the angle and time series data of the acceleration.

13. The information processing device according to claim 12, wherein whether or not the user is in the pedaling state is determined based on a first similarity degree between the time series data of the angle and the time series data of the acceleration.

14. The information processing device according to claim 13, wherein the first similarity degree includes a correlation coefficient between the time series data of the angle and the time series data of the acceleration.

15. The information processing device according to claim 12, wherein whether or not the user is in the pedaling state is determined further based on a frequency spectrum of the angle and a frequency spectrum of the acceleration acquired by transforming the time series data of the angle and the time series data of the acceleration into a frequency domain.

16. The information processing device according to claim 15, wherein whether or not the user is in the pedaling state is determined based on a second similarity degree between the frequency spectrum of the angle and the frequency spectrum of the acceleration.

17. The information processing device according to claim 16, wherein the second similarity degree includes a correlation coefficient between the frequency spectrum of the angle and the frequency spectrum of the acceleration.

18. The information processing device claim 16, wherein the user is determined to be in the pedaling state in a case where a first similarity degree between the time series of the angle and the time series data of the acceleration is greater than a first threshold value and a second similarity degree between the frequency spectrum of the angle and the frequency spectrum of the acceleration is greater than a second threshold value.

19-25. (canceled)

26. An energy calculation system comprising:
a memory configured to store instructions; and
a processor configured to execute the instructions to calculate energy consumed by the user by riding the bicycle based on a length of the pedaling period or the non-pedaling time acquired by the information processing device according to claim 1.

27. An information processing method comprising:
acquiring action information of a user;
extracting a plurality of pedaling periods during which the user pedals a bicycle from the action information; and
calculating a non-pedaling time during which the user is on the bicycle and does not pedal based on a start time of each of the plurality of pedaling periods and an end time of each of the plurality of pedaling periods.

28. (canceled)
